(19) United States
(12) Patent Application Publication
(10) Pub. No.: US 2019/0309522 A1
(43) Pub. Date: Oct. 10, 2019

(54) WALL RAIL SECTION FORMING AN ODOUR NEUTRALISING STRUCTURE AND FALSE WALL COMPRISING SUCH A WALL RAIL SECTION

(71) Applicant: NORMALU, KEMBS (FR)

(72) Inventor: Jean-Marc SCHERRER, RIEDISHEIM (FR)

(21) Appl. No.: 16/466,431

(22) PCT Filed: Dec. 4, 2017
(86) PCT No.: PCT/FR2017/053367

§ 371 Date: Jun. 4, 2019

(30) Foreign Application Priority Data
Dec. 6, 2016 (FR) FR 16 61976

(51) Int. Cl. E04F 13/00; E04B 9/30; A61L 9/014; A61L 9/12; A61L 9/04

(52) U.S. Cl. CPC E04F 13/005; E04B 9/303; A61L 9/014; A61L 2209/15; A61L 9/046; A61L 2209/22; A61L 9/12

(57) ABSTRACT

An odour neutralising structure comprising a wall rail section arranged to hang a stretched fabric in order to produce a false wall and at least one filter cartridge containing a sorbent material that can absorb one or more specific odour(s), said cartridge being housed in a groove in the wall rail section.

### BACKGROUND

The invention relates to the treatment of odours qualified as “bad” in a closed (local) environment.

The invention more particularly relates to a structure for neutralizing ambient odours qualified as bad smells. In this application, the term “neutralization” also includes the terms “reduction”, “limitation” and “elimination” of odours.

Different causes can generate bad smells in a room. In a residential area, it may be tobacco, food, bacteria, moulds, etc. Bad smells in a room are usually treated according to their cause, either by removing the source of the bad smell itself, or by masking it by diffusion of a masking agent, or by reducing it with a substance adapted to absorb/adsorb the bad smell.

For odour masking or absorption/adsorption, it is common to use fragrant substance diffusers or containers containing compositions with odour absorption/adsorption properties. The disadvantage of diffusers and absorption/adsorption containers is their support. The latter are classically arranged on tables, furniture or equivalent. Depending on the destination of the room, it may be that such supports are not always present. In addition, the visual presence of such products may in some cases be unpleasant or even inappropriate.

The invention aims to remedy these problems by proposing an odour neutralising structure placed in a room without interfering with the visual aspect of the room and this independently of any arrangement or furniture in the room.

### SUMMARY

For this purpose, and according to a first aspect, the invention proposes an odour neutralising structure comprising a wall rail section arranged to allow the hanging of a stretched fabric for the production of a false wall and at least one filter cartridge containing a sorbent material that can absorb one or more specific volatile smells (odours), said cartridge being housed in a groove in the wall rail section.

By equipping wall rail sections with one or more filter cartridge(s) as described above, it is possible to install false shells (false walls, false ceilings, decorative walls) having the function of neutralizing odours throughout the room, in addition to their primary function of hiding technical or decorative elements. Thus, whatever the size of the room and whatever the interior arrangement of the room, odour neutralisation is ensured without altering the visual appearance of the room, the filter cartridges being invisible from the room.

Advantageously, the sorbent material includes zeolite.

Advantageously, the filter cartridge also contains a fragrant substance, said cartridge being arranged to release said substance.

Advantageously, the groove has an aperture opening above the inner face of the fabric when the latter is attached to the wall rail section. The inner face of the fabric means the face of the fabric opposite the inside of the room when the fabric hangs from the wall rail section attached to a wall of the room. In contrast, the outer face of the fabric refers to the face facing inside the room.

Advantageously, the groove has an aperture opening in the lower part of the wall rail section. This configuration has the advantage of facilitating access to the groove containing the filter cartridge and thus facilitating the installation, removal or replacement of the cartridge.

Advantageously, the groove has an aperture opening into a space delimited by the inner face of the said fabric when the latter is attached to the wall rail section.

Advantageously, the wall rail section has a flap giving access to the groove

Advantageously, the groove is closed by a cover with holes for the passage of volatile compounds.

The invention also relates to a wall rail section forming an odour neutralising structure as described above.

The invention also relates to a false wall comprising a fabric stretched over a rail formed by at least one wall rail section constituting an odour neutralising structure as described above.

According to a particular configuration, the fabric is micro-perforated. This has the advantage of allowing the neutralization of odours and/or the passage of the fragrant substance also in the space delimited by the inner face of the fabric.

Advantageously, the groove in which the filter cartridge is attached is a groove accessible from the room in which the false wall is mounted. This makes the filter cartridge accessible from the room without having to remove the fabric.

### BRIEF DESCRIPTION OF THE DRAWINGS

Other objects and advantages of the invention will appear in the following description, with reference to the attached drawings, in which:

FIG. 1 shows a schematic cross-sectional view of a wall rail section forming an odour neutralising structure according to a first exemplary embodiment of the invention, a stretched fabric being shown hanging from said rail section;

FIG. 2 shows an alternative embodiment of the wall rail section of FIG. 1.

FIG. 3 shows an alternative embodiment of the wall rail section of FIG. 2.

FIG. 4 shows a schematic cross-sectional view of a wall rail section forming an odour neutralising structure according to a second exemplary embodiment of the invention, a stretched fabric being shown hanging from said rail section;

FIGS. 5a and 5b shows an alternative embodiment of the wall rail section of FIG. 4.

For greater clarity, the same or similar elements of the different embodiments are marked by identical references on all the figures.

### DETAILED DESCRIPTION

The wall rail sections shown in the figures can be used as a wall rail section intended to be attached to a wall shell to form, together with a stretched fabric, a false ceiling or a frame section. The figures show a wall rail section in the attached position on a wall shell.

FIG. 1 shows a first exemplary embodiment of a wall rail section 1 on which a stretched fabric is hung. In the example shown, the wall rail section 1 has a first vertical wing 3 for attaching the wall rail section 1 to a wall (not shown), a first groove 6 arranged to allow the fabric 2 to hang and a second groove 7 arranged between the first groove 6 and the first wing 3.

The first and second grooves 6, 7 have a downward aperture respectively. Both are laterally delimited by a common wall 4. In the embodiment shown, the common wall 4 extends parallel to the first wing 3. The wall rail section 1 has a second wing 5 parallel to the common wall 4. The first groove 6 is thus laterally delimited by the second wing 5 and a portion of the common wall 14. In the embodiment shown, the first wing 3, the common wall 4 and the second wing 5 are parallel to each other.

The wall rail section can be equipped with one or more filter cartridge(s) to filter odours from the room. To do this, the filter cartridge contains a sorbent material such as zeolite. It is of course obvious that the sorbent material will be chosen according to the type of odours that are to be removed from the room. In addition to the sorbent material, the filter cartridge may also contain a fragrant substance. Said cartridge will then be arranged to ensure the release of the fragrant substance. Thus, in addition to the absorption or adsorption of bad odours, a fragrant volatile compound may be diffused into the room, with said cartridge being arranged to release said substance.

In the embodiment shown, the filter cartridge 9 is housed in the second groove 7. In the example given, it is attached on the common wall 4. In the case of cartridges that also diffuse fragrant substances, the fragrant substance will be diffused directly from the groove of the wall rail section 1 towards the inside of the room.

In order to avoid the unsightly appearance of the cartridge 9, the common wall 4 is provided at its lower end with a shoulder 11 extending towards the inside of the second groove 7. The shoulder is such that it is long enough to hide the cartridge 9 to a person in the room under the false ceiling.

In the embodiment shown, the shoulder 11 is provided at the lower end of the common wall 4. It also has an extension 12 extending towards the inside of the first groove 6, said extension 12 forming a shoulder for the attachment of a harpoon 20 as shown or any other equivalent means equipping the edge of the fabric 2. The shoulder 11 and the extension 12 extend substantially perpendicularly to the common wall 4.

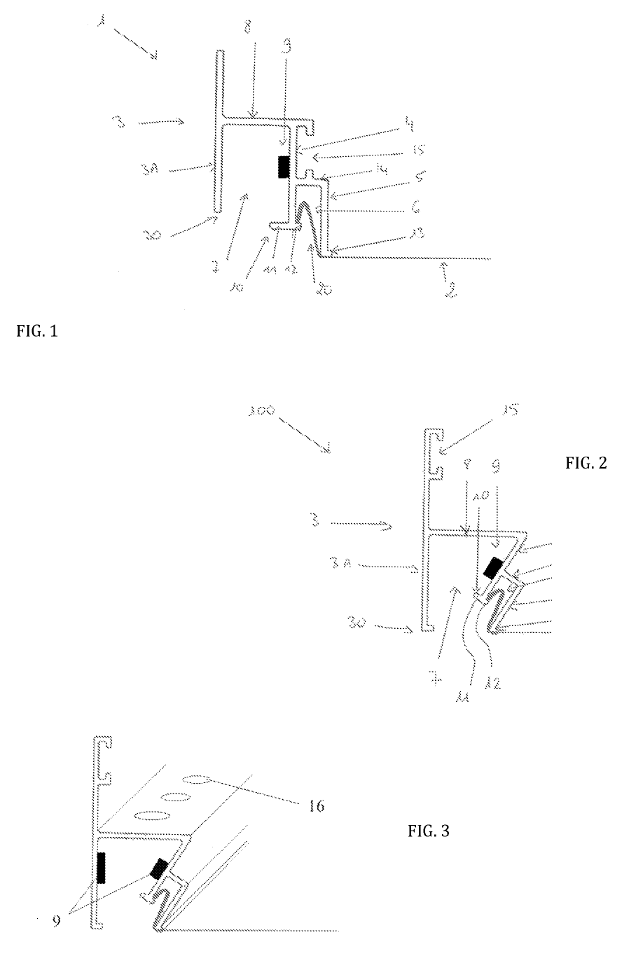

FIG. 2 illustrates an alternative embodiment of the rail section 1 according to the invention. In this alternative embodiment, the wall rail section, referenced 100, differs from the one previously described, in particular by the orientation of the common wall 4 and the second wing 5.

In particular, the common wall 4 and the second wing 3 are arranged to converge towards the lower portion 3A of the first wing 3. This has the advantage of improving the hiding of the filter cartridge 9 as well as the hiding of the hanging elements of the fabric 2 on the rail 100 (the harpoon 20 or equivalent means).

Advantageously, the second wing 5 has a length such that its free end (the end bearing the fabric support tab) is located at the same level as the free end 30 of the lower portion 3A of the first wing. The common wall 4 is sized to have an end that stops more than the lower end 30 of the lower portion 3A of the first wing 3.

Advantageously, the common wall 4 has a length such that the extension 12 and the free end of the second wing 5 are aligned substantially vertically. This configuration has the advantage of making the harpoon invisible to a person placed under the false ceiling, and in particular directly under the rail.

In addition, in this alternative embodiment, the rail 15 for connecting the wall rail section with another wall rail section is carried by the portion of the first wing 3 above the web 8.

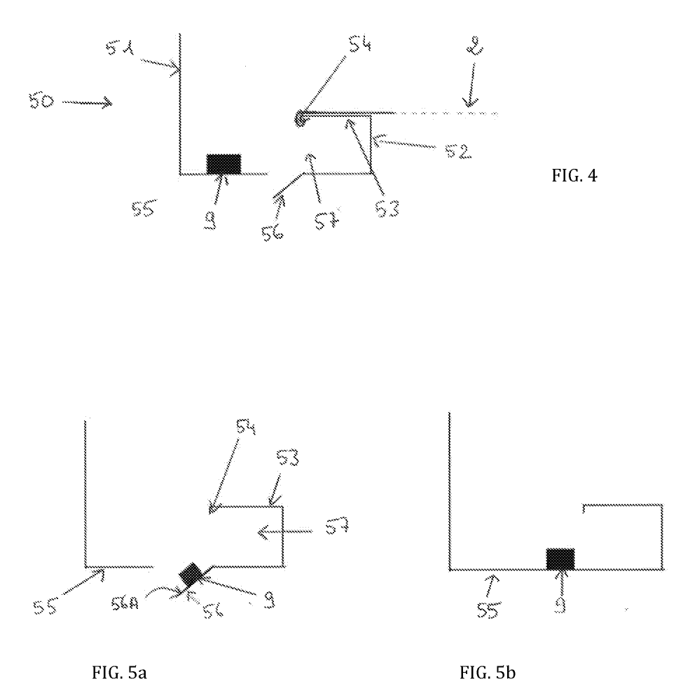

FIG. 4 illustrates another exemplary embodiment of a wall rail section 50 constituting an odour neutralising structure. In this exemplary embodiment, the wall rail section 50 includes a first and a second wing 51, 52 parallel to each other, one (here wing 52) being intended to be attached on a wall (not shown). The wall rail section 50 also has an arm 53 extending perpendicularly from the wing 52 towards the wing 51. The arm 53 is provided at the free end with a shoulder 54 arranged to allow the stretched fabric 2 to be hung as shown in FIG. 3. The wings 51, 52 are connected to each other in the lower part by a connecting wall 55 (also called the lower wall). The wings 51, 52 define with the lower wall 55 a groove 57 opening into the space above the stretched fabric 2 (velum) when it is hung on the wall rail section 50. The filter cartridge 9 is located inside the groove 57. In the embodiment shown, it is placed on the lower wall 55 which forms the bottom of the groove.

In order to allow the passage of volatile fumes/compounds from the room into the velum and, if necessary, the diffusion of fragrant volatile substances within the room when the filter cartridge combines the filter and diffuser functions, the fabric 2 used is advantageously micro-perforated.

In order to facilitate the replacement of the filter cartridge 9, and in particular to avoid removing the fabric 2 to remove or replace the cartridge, the wall rail section 50 advantageously includes an access flap 56. In the embodiment shown, the access flap 56 is provided at the lower wall 55. This allows easy access to the inside of the groove 57 and allows the filter cartridge 9 to be inserted, removed or replaced without having to remove the fabric 2. In order to improve the passage of odours to be neutralised and present inside the room and the diffusion of fragrant compounds when the filter cartridge 9 contains such compounds, the lower wall 9 and/or the access flap 9 may be provided with orifices.

FIGS. 5a and 5b illustrate an alternative embodiment of FIG. 4, with FIG. 5a showing the access flap 56 at the groove 57 in the open position and FIG. 5b showing the access flap in the closed position. In this embodiment, the filter cartridge 9 is attached on the access flap 56, on the inner face 56A of the latter. The inner face of the flap means the face oriented towards the inside of the groove when the access flap 56 is in the closed position. The filter cartridge 9 can be attached on the flap by gluing or any other means known to the skilled person. The inner face 56A of the flap can be provided with retaining means, such as lugs, defining a location in which the cartridge is placed and forced in or by clipping.

The advantage of arranging the wall rail sections with the cartridge shown in FIGS. 4 and 5a and 5b is that it allows the neutralisation of odours also present within the space delimited by the inner face of the stretched fabric (the velum in the case of a false ceiling).

Advantageously, the section may have passage apertures 16 to allow air to pass between the plenum (the space behind the fabric 2) and the room. FIG. 3 shows, for example, the section of FIG. 2 with such apertures. It is of course obvious that such apertures can be provided on any type of hanging section according to the invention. In the example shown, air passage apertures 16 are carried by the web 8. However, it is understood that the location of these apertures will be defined according to the shape of the section but also to the position of the filter cartridges. It is indeed advantageous to provide for the passage apertures to be located in such a way as to allow the passage of air circulating between the plenum and the room through the groove housing a filter cartridge. In the embodiment shown, the passage apertures 16 define circular-shaped windows. This is of course one exemplary embodiment, and the apertures can have a different shape and/or arrangement. Besides, the number of apertures is variable.

In the examples described above, the hanging sections may include a single cartridge. However, the hanging section may contain several cartridges, which may be carried on the same wall or on different walls (FIG. 3).

The invention is described above by way of example. It is understood that the person skilled in the art is able to carry out different embodiments of the invention without departing from the scope of the invention. In particular, the invention is not limited to the arrangement nor to the wall rail sections illustrated in the figures above. In addition, a wall rail section may be provided, which comprises several filter cartridges arranged in a single groove or in separate grooves.

1. An odour neutralising structure comprising a wall rail section arranged to hang a stretched fabric in order to produce a false wall and at least one filter cartridge containing a sorbent material that can absorb one or more specific odour(s), said cartridge being housed in a groove in the wall rail section.

2. The odour neutralising structure according to claim 1, wherein the sorbent material comprises zeolite.

3. The odour neutralising structure according to claim 1, wherein the filter cartridge further contains a fragrant substance, said cartridge being arranged to release said substance.

4. The odour neutralising structure according to claim 1, wherein the groove has an aperture opening above the inner face of the fabric when the latter is attached to the wall rail section.

5. The odour neutralising structure according to claim 1, wherein the groove has an aperture opening in the lower part of the wall rail section.

6. The odour neutralising structure according to claim 1, wherein the groove has an aperture opening into a space delimited by the inner face of said stretched fabric when the latter is attached to the wall rail section.

7. The odour neutralising structure according to claim 1, wherein the wall rail section has a flap giving access to the groove.

8. The odour neutralising structure according to claim 1, wherein the groove is closed by a cover provided with holes for the passage of volatile compounds.

9. A wall rail section comprising, with one or more filter cartridge(s) containing a sorbent material that can absorb one or more specific odour(s) housed in a groove of the wall rail section, an odour neutralising structure according to claim 1.

10. A false wall comprising a stretched fabric on a rail section formed by at least one wall rail section according to claim 9.

11. The false wall according to claim 10, wherein the stretched fabric is micro-perforated.

12. The false wall according to claim 11, wherein the groove which the filter cartridge is attached is a groove accessible from a room in which the false wall is mounted.
